(19) United States
(12) Patent Application Publication
(10) Pub. No.: US 2023/0233227 A1
(43) Pub. Date: Jul. 27, 2023

(54) Scalpel Blades, Scalpels, Scalpel Assemblies, and Methods Thereof

(71) Applicant: Bard Access Systems, Inc., Salt Lake City, UT (US)

(72) Inventors: Eric W. Lindekugel, Salt Lake City, UT (US); Glade H. Howell, Draper, UT (US)

(21) Appl. No.: 18/098,607

(22) Filed: Jan. 18, 2023

Related U.S. Application Data
(60) Provisional application No. 63/301,869, filed on Jan. 21, 2022.

(51) Int. Cl. A61B 17/3213; A61B 17/3209

(52) U.S. Cl. CPC A61B 17/3213; A61B 17/32093; A61B 2017/00477

(57) ABSTRACT

Scalpel blades, scalpels, scalpel assemblies, and methods thereof can be configured to reduce or even eliminate occurrences of skin bridges from skin-nicking such as when expanding insertion sites for catheters. For example, a scalpel blade can include a blade edge and a back edge, which back edge can include a pair of guidewire clips configured to clip onto a guidewire. The blade edge can terminate with a blade tip at a distal end of the scalpel blade. The pair of guidewire clips can be configured to clip onto the guidewire with sufficient clearance for slidably guiding the scalpel blade along the guidewire when skin-nicking a patient's skin and fascia around an insertion site established by a percutaneous puncture. The scalpels and scalpel assemblies can include the example scalpel blade. The methods can include methods of using any of the scalpel blades, scalpels, or scalpel assemblies disclosed herein.

### PRIORITY

This application claims the benefit of priority to U.S. Provisional Application No. 63/301,869, filed Jan. 21, 2022, which is incorporated by reference in its entirety into this application.

### BACKGROUND

Insertion of central venous catheters (“CVCs”) into patients is a multi-step process. For example, after a percutaneous puncture with a needle at a chosen insertion site, whereby a needle tract is established from an area of skin to a blood-vessel lumen, a guidewire is inserted through the needle into the blood-vessel lumen. Subsequently, the needle is removed leaving the guidewire in place. At this point, a scalpel is typically used to perform a skin nick in which both skin and facia are cut about the insertion site to ease insertion of the CVC. Such a skin nick is performed with a traditional scalpel by placing a back edge of a scalpel blade against the guidewire and running a blade edge of the scalpel blade into the skin and fascia to make a larger hole. However, it takes a great deal of skill and practice to make a clean skin nick with a such a scalpel. Indeed, the back edge of the scalpel blade must be firmly held against the guidewire without lifting the scalpel blade therefrom to ensure the skin and facia immediately extending away from the guidewire are cut instead of that about a periphery of the guidewire; otherwise, another percutaneous puncture with a so-called skin bridge between it and the percutaneous puncture established by the needle will result, which can catch between the guidewire and a catheter tip of the CVC and cause unnecessary trauma. A skin-nicking means and method to facilitate clean skin-nicking about an insertion site is needed for at least the insertion of CVCs into patients in view of the foregoing.

Disclosed herein are scalpel blades, scalpels, scalpel assemblies, and methods thereof that address at least the foregoing need.

SUMMARY

Disclosed herein is scalpel blade including, in some embodiments, a blade edge and a back edge including a pair of guidewire clips configured to clip onto a guidewire. The blade edge terminates with a blade tip at a distal end of the scalpel blade. The pair of guidewire clips are configured to clip onto the guidewire with sufficient clearance for slidably guiding the scalpel blade along the guidewire when skin-nicking a patient's skin and fascia around an insertion site established by a percutaneous puncture.

In some embodiments, the pair of guidewire clips are disposed under the back edge of the scalpel blade or along a side of the back edge of the scalpel blade.

In some embodiments, each clip of the pair of guidewire clips includes an opening that opens toward the back edge of the scalpel blade and away from a side of the scalpel blade opposite the other clip of the pair of guidewire clips.

In some embodiments, each clip of the pair of guidewire clips is approximately a three-quarter sector of a circle in transverse cross section.

In some embodiments, each clip of the pair of guidewire clips includes an opening that opens along a side of the scalpel blade or opposite thereto on a same side of the scalpel blade. Each clip of the pair of guidewire clips is mutually exclusive of the other clip of the pair of guidewire clips with respect to the opening thereof.

In some embodiments, each clip of the pair of guidewire clips is approximately a semicircle in transverse cross section.

In some embodiments, each clip of the pair of guidewire clips is sufficiently separated along the back edge of the scalpel blade from the other clip of the pair of guidewire clips to allow the guidewire to easily cross over the back edge of the scalpel blade without appreciably bending the guidewire when loading the guidewire into each clip of the pair of guidewire clips.

In some embodiments, the guidewire is coaxial with the pair of guidewire clips when the guidewire is loaded therein.

In some embodiments, the guidewire is sufficiently coaxial with the pair of guidewire clips for rotating the scalpel blade around the guidewire to a desired rotational angle for the skin-nicking of the patient's skin and fascia around the insertion site.

In some embodiments, the scalpel blade further includes a handle-attachment slot configured for attaching a scalpel handle to the scalpel blade to form a scalpel.

Also disclosed herein is a scalpel including, in some embodiments, a scalpel handle and a scalpel blade coupled to the scalpel handle. The scalpel blade includes a blade edge and a back edge including a pair of guidewire clips configured to clip onto a guidewire. The blade edge terminates with a blade tip at a distal end of the scalpel blade. The pair of guidewire clips are configured to clip onto the guidewire with sufficient clearance for slidably guiding the scalpel along the guidewire when skin-nicking a patient's skin and fascia around an insertion site established by a percutaneous puncture.

In some embodiments, the scalpel handle includes a blade-attachment fixture, and the scalpel blade further includes a handle-attachment slot. The blade-attachment fixture is disposed in the handle-attachment slot coupling the scalpel blade to the scalpel handle.

In some embodiments, the pair of guidewire clips are disposed under the back edge of the scalpel blade or along a side of the back edge of the scalpel blade.

In some embodiments, each clip of the pair of guidewire clips includes an opening that opens toward the back edge of the scalpel blade and away from a side of the scalpel blade opposite the other clip of the pair of guidewire clips.

In some embodiments, each clip of the pair of guidewire clips is approximately a three-quarter sector of a circle in transverse cross section.

In some embodiments, each clip of the pair of guidewire clips includes an opening that opens along a side of the scalpel blade or opposite thereto on a same side of the scalpel blade. Each clip of the pair of guidewire clips is mutually exclusive of the other clip of the pair of guidewire clips with respect to the opening thereof.

In some embodiments, each clip of the pair of guidewire clips is approximately a semicircle in transverse cross section.

In some embodiments, each clip of the pair of guidewire clips is sufficiently separated along the back edge of the scalpel blade from the other clip of the pair of guidewire clips to allow the guidewire to easily cross over the back edge of the scalpel blade without appreciably bending the guidewire when loading the guidewire into each clip of the pair of guidewire clips.

In some embodiments, the guidewire is coaxial with the pair of guidewire clips when the guidewire is loaded therein.

In some embodiments, the guidewire is sufficiently coaxial with the pair of guidewire clips for rotating the scalpel around the guidewire to a desired rotational angle for the skin-nicking of the patient's skin and fascia around the insertion site.

Also disclosed herein is a scalpel assembly including, in some embodiments, a guidewire, a scalpel handle, and a scalpel blade coupled to the scalpel handle to form a scalpel. The scalpel blade includes a blade edge and a back edge including a pair of guidewire clips clipped onto the guidewire. The blade edge terminates with a blade tip at a distal end of the scalpel blade. The pair of guidewire clips are clipped onto the guidewire with sufficient clearance for slidably guiding the scalpel along the guidewire when skin-nicking a patient's skin and fascia around an insertion site established by a percutaneous puncture.

In some embodiments, the scalpel handle includes a blade-attachment fixture, and the scalpel blade further includes a handle-attachment slot. The blade-attachment fixture is disposed in the handle-attachment slot coupling the scalpel blade to the scalpel handle.

In some embodiments, the pair of guidewire clips are disposed under the back edge of the scalpel blade or along a side of the back edge of the scalpel blade.

In some embodiments, each clip of the pair of guidewire clips includes an opening that opens toward the back edge of the scalpel blade and away from a side of the scalpel blade opposite the other clip of the pair of guidewire clips.

In some embodiments, each clip of the pair of guidewire clips is approximately a three-quarter sector of a circle in transverse cross section.

In some embodiments, each clip of the pair of guidewire clips includes an opening that opens along a side of the scalpel blade or opposite thereto on a same side of the scalpel blade. Each clip of the pair of guidewire clips is mutually exclusive of the other clip of the pair of guidewire clips with respect to the opening thereof.

In some embodiments, each clip of the pair of guidewire clips is approximately a semicircle in transverse cross section.

In some embodiments, each clip of the pair of guidewire clips is sufficiently separated along the back edge of the scalpel blade from the other clip of the pair of guidewire clips to allow the guidewire to easily cross over the back edge of the scalpel blade without appreciably bending the guidewire when loading the guidewire into each clip of the pair of guidewire clips.

In some embodiments, the guidewire is coaxial with the pair of guidewire clips when the guidewire is loaded therein.

In some embodiments, the guidewire is sufficiently coaxial with the pair of guidewire clips for rotating the scalpel around the guidewire to a desired rotational angle for the skin-nicking of the patient's skin and fascia around the insertion site.

Also disclosed is a method for inserting a catheter into a blood-vessel lumen. The method includes, in some embodiments, a needle tract-establishing step, a guidewire-inserting step, a needle-withdrawing step, a skin-nicking step, a scalpel blade-removing step, and a catheter-inserting step. The needle tract-establishing step includes establishing a needle tract from an area of skin to the blood-vessel lumen of a patient with a needle at a chosen insertion site for inserting the catheter. The guidewire-inserting step includes inserting a guidewire through the needle into the blood-vessel lumen. The needle-withdrawing step includes withdrawing the needle from the needle tract leaving the guidewire in place in the blood-vessel lumen. The skin-nicking step includes skin-nicking skin and fascia of the patient around the insertion site with a scalpel blade guided along the guidewire. The scalpel blade includes a blade edge and a back edge including a pair of guidewire clips clipped onto the guidewire. The blade edge terminates with a blade tip at a distal end of the scalpel blade. The pair of guidewire clips are clipped onto the guidewire with sufficient clearance for slidably guiding the scalpel blade along the guidewire during the skin-nicking step. The scalpel blade-removing step includes removing the scalpel blade from the guidewire. The catheter-inserting step includes inserting the catheter into the blood-vessel lumen over the guidewire.

In some embodiments, the method further includes a guidewire-loading step. The guidewire-loading step includes loading the guidewire into the pair of guidewire clips of the scalpel blade by rotating the scalpel blade and placing the guidewire in an opening of each clip of the pair of guidewire clips for each clip of the pair of guidewire clips.

In some embodiments, the opening of each clip of the pair of guidewire clips opens toward the back edge of the scalpel blade and away from a side of the scalpel blade opposite the other clip of the pair of guidewire clips.

In some embodiments, each clip of the pair of guidewire clips is approximately a three-quarter sector of a circle in transverse cross section.

In some embodiments, the opening of each clip of the pair of guidewire clips opens along a side of the scalpel blade or opposite thereto on a same side of the scalpel blade. Each clip of the pair of guidewire clips is mutually exclusive of the other clip of the pair of guidewire clips with respect to the opening thereof.

In some embodiments, each clip of the pair of guidewire clips is approximately a semicircle in transverse cross section.

In some embodiments, the scalpel blade-removing step includes unloading the guidewire from the pair of guidewire clips of the scalpel blade by rotating the scalpel blade and extracting the guidewire from the opening of each clip of the pair of guidewire clips for each clip of the pair of guidewire clips.

In some embodiments, the scalpel blade-removing step includes withdrawing the scalpel blade over a proximal end of the guidewire.

In some embodiments, the method further includes a scalpel-forming step. The scalpel-forming step includes coupling the scalpel blade to a scalpel handle to form a scalpel by inserting a blade-attachment fixture of the scalpel handle into a handle-attachment slot of the scalpel blade.

In some embodiments, the method further a scalpel blade-rotating step. The scalpel blade-rotating step includes rotating the scalpel blade or scalpel around the guidewire to a desired rotational angle for the skin-nicking of the skin and fascia of the patient around the insertion site. The guidewire is sufficiently coaxial with the pair of guidewire clips for the rotating of the scalpel or scalpel around the guidewire.

These and other features of the concepts provided herein will become more apparent to those of skill in the art in view of the accompanying drawings and following description, which describe particular embodiments of such concepts in greater detail.

### DRAWINGS

FIG. 1 illustrates a scalpel blade while clipped onto a guidewire with a first pair of guidewire clips in accordance with some embodiments.

FIG. 2 illustrates a distal end of the scalpel blade while clipped onto a guidewire with the first pair of guidewire clips in accordance with some embodiments.

FIG. 3 illustrates a perspective view from a proximal end of the scalpel blade while clipped onto a guidewire with the first pair of guidewire clips in accordance with some embodiments.

FIG. 4 illustrates a perspective view from a distal end of the scalpel blade while clipped onto a guidewire with the first pair of guidewire clips in accordance with some embodiments.

FIG. 5 illustrates a detailed view from a side of the scalpel blade while clipped onto a guidewire with the first pair of guidewire clips in accordance with some embodiments.

FIG. 6 illustrates the scalpel blade with a second pair of guidewire clips in accordance with some embodiments.

FIG. 7A illustrates a simplified distal end-on view of the scalpel blade and a distal guidewire clip of the first pair of guidewire clips in accordance with some embodiments.

FIG. 7B illustrates a simplified proximal end-on view of the scalpel blade and a proximal guidewire clip of the first pair of guidewire clips in accordance with some embodiments.

FIG. 8A illustrates a simplified distal end-on view of the scalpel blade and the distal guidewire clip of the first pair of guidewire clips in accordance with some other embodiments.

FIG. 8B illustrates a simplified proximal end-on view of the scalpel blade and the proximal guidewire clip of the first pair of guidewire clips in accordance with some other embodiments.

FIG. 9A illustrates a simplified distal end-on view of the scalpel blade and a distal guidewire clip of the second pair of guidewire clips in accordance with some embodiments.

FIG. 9B illustrates a simplified proximal end-on view of the scalpel blade and a proximal guidewire clip of the second pair of guidewire clips in accordance with some embodiments.

FIG. 10 illustrates a scalpel including the scalpel blade coupled to a scalpel handle with the scalpel blade clipped onto a guidewire with the first pair of guidewire clips in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or “proximal section” of, for example, a scalpel includes a portion or section of the scalpel intended to be near a clinician when the scalpel is used on a patient. Likewise, a “proximal length” of, for example, the scalpel includes a length of the scalpel intended to be near the clinician when the scalpel is used on the patient. A “proximal end” of, for example, the scalpel includes an end of the scalpel intended to be near the clinician when the scalpel is used on the patient. The proximal portion, the proximal section, or the proximal length of the scalpel can include the proximal end of the scalpel; however, the proximal portion, the proximal section, or the proximal length of the scalpel need not include the proximal end of the scalpel. That is, unless context suggests otherwise, the proximal portion, the proximal section, or the proximal length of the scalpel is not a terminal portion or terminal length of the scalpel.

With respect to “distal,” a “distal portion” or a “distal section” of, for example, a scalpel includes a portion or section of the scalpel intended to be near or on a patient when the scalpel is used on the patient. Likewise, a “distal length” of, for example, the scalpel includes a length of the scalpel intended to be near or on the patient when the scalpel is used on the patient. A “distal end” of, for example, the scalpel includes an end of the scalpel intended to be near or on the patient when the scalpel is used on the patient. The distal portion, the distal section, or the distal length of the scalpel can include the distal end of the scalpel; however, the distal portion, the distal section, or the distal length of the scalpel need not include the distal end of the scalpel. That is, unless context suggests otherwise, the distal portion, the distal section, or the distal length of the scalpel is not a terminal portion or terminal length of the scalpel.

Unless defined otherwise, all technical and scientific terms used herein have the same meaning as commonly understood by those of ordinary skill in the art.

As set forth above, the insertion of CVCs into patients is a multi-step process. For example, after a percutaneous puncture with a needle at a chosen insertion site, whereby a needle tract is established from an area of skin to a blood-vessel lumen, a guidewire is inserted through the needle into the blood-vessel lumen. Subsequently, the needle is removed leaving the guidewire in place. At this point, a scalpel is typically used to perform a skin nick in which both skin and facia are cut about the insertion site to ease insertion of the CVC. Such a skin nick is performed with a traditional scalpel by placing a back edge of a scalpel blade against the guidewire and running a blade edge of the scalpel blade into the skin and fascia to make a larger hole. However, it takes a great deal of skill and practice to make a clean skin nick with a such a scalpel. Indeed, the back edge of the scalpel blade must be firmly held against the guidewire without lifting the scalpel blade therefrom to ensure the skin and facia immediately extending away from the guidewire are cut instead of that about a periphery of the guidewire; otherwise, another percutaneous puncture with a so-called skin bridge between it and the percutaneous puncture established by the needle will result, which can catch between the guidewire and a catheter tip of the CVC and cause unnecessary trauma. A skin-nicking means and method to facilitate clean skin-nicking about an insertion site is needed for at least the insertion of CVCs into patients in view of the foregoing.

Disclosed herein are scalpel blades, scalpels, scalpel assemblies, and methods thereof for reducing or even eliminating occurrences of skin bridges from skin-nicking such as when expanding insertion sites for catheters. For example, a scalpel blade can include a blade edge and a back edge, which back edge can include a pair of guidewire clips configured to clip onto a guidewire. The blade edge can terminate with a blade tip at a distal end of the scalpel blade. The pair of guidewire clips can be configured to clip onto the guidewire with sufficient clearance for slidably guiding the scalpel blade along the guidewire when skin-nicking a patient's skin and fascia around an insertion site established by a percutaneous puncture. The scalpels and scalpel assemblies can include the example scalpel blade. The methods can include methods of using any of the scalpel blades, scalpels, or scalpel assemblies disclosed herein.

### Scalpel Blades

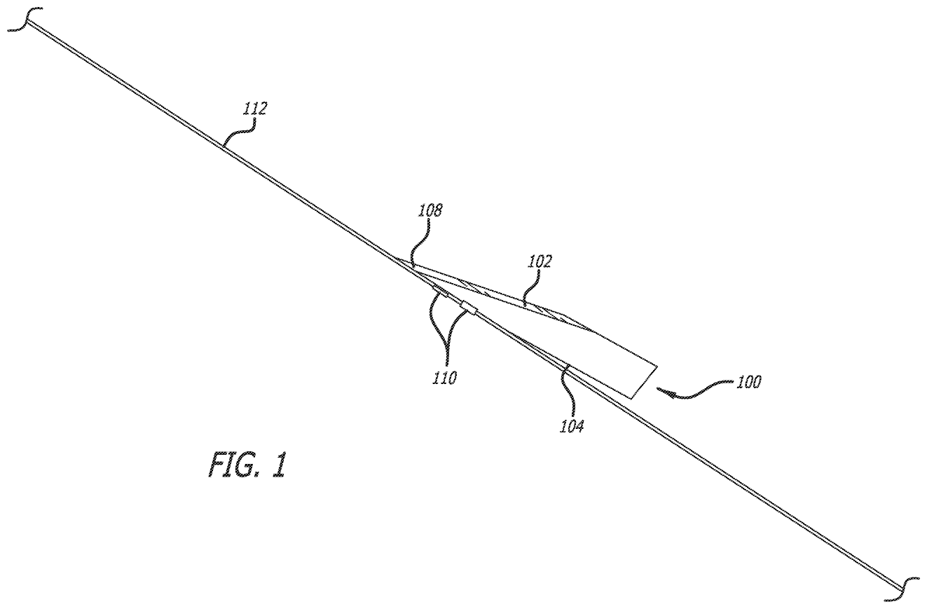

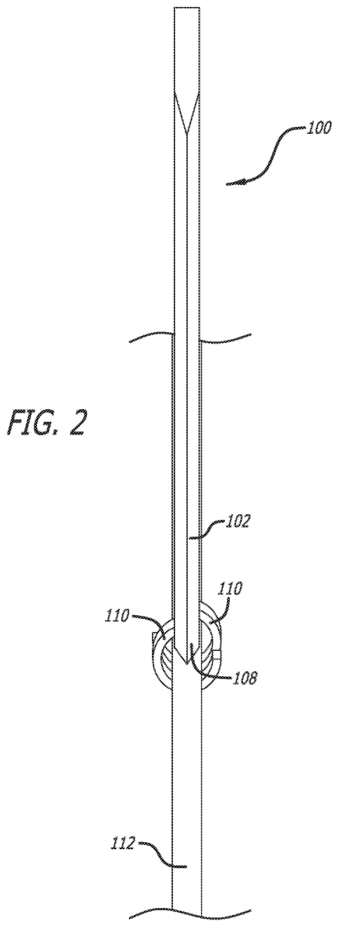

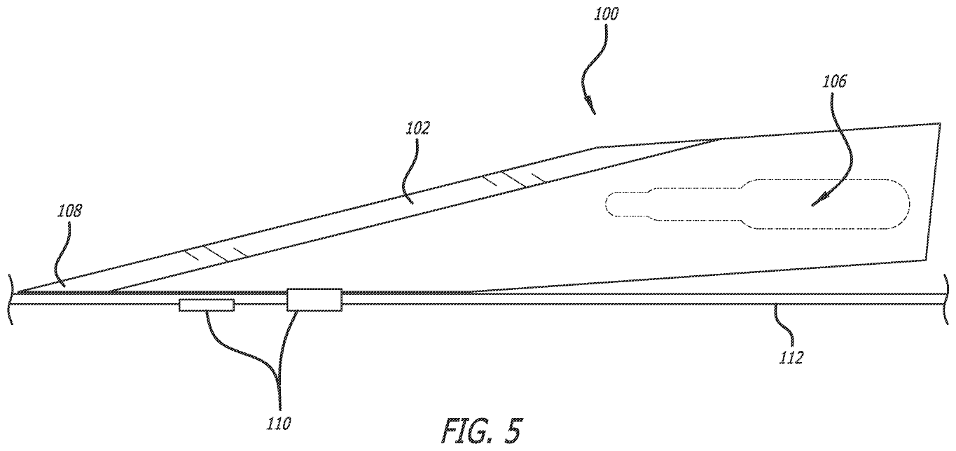

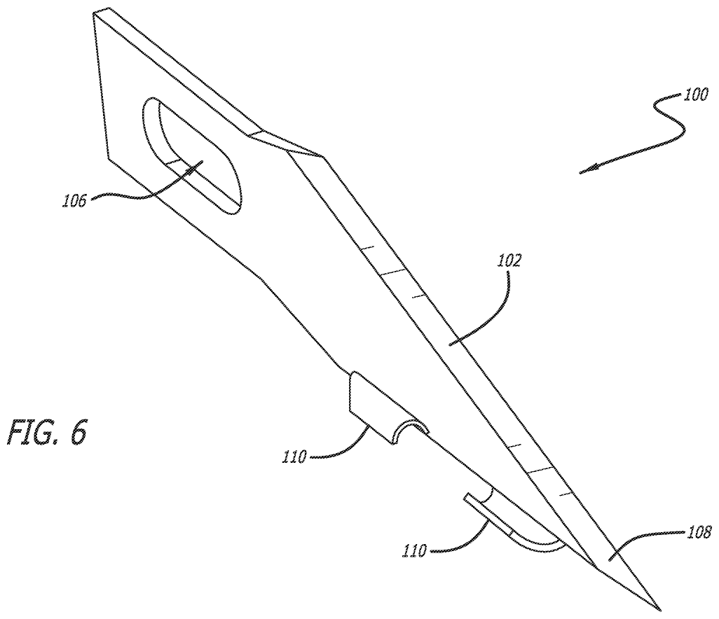

FIGS. 1-6, 7A, 7B, 8A, 8B, 9A, and 9B illustrate various views of a scalpel blade 100 in accordance with some embodiments.

As shown, the scalpel blade 100 includes a sharp or blade edge 102 and a dull or back edge 104. Optionally, the scalpel blade 100 further includes a handle-attachment slot 106. Such a scalpel blade can be formed of surgical steel.

The blade edge 102 terminates with a blade tip 108 at a distal end of the scalpel blade 100.

The back edge 104 includes a pair of guidewire clips 110 configured to clip onto a guidewire 112. However, it should be understood the back edge 104 is not limited to the pair of guidewire clips 110. Indeed, the back edge 104 can include a single guidewire clip or a set of three or more guidewire clips with optionally alternating openings like those of the pair of guidewire clips 110 set forth below.

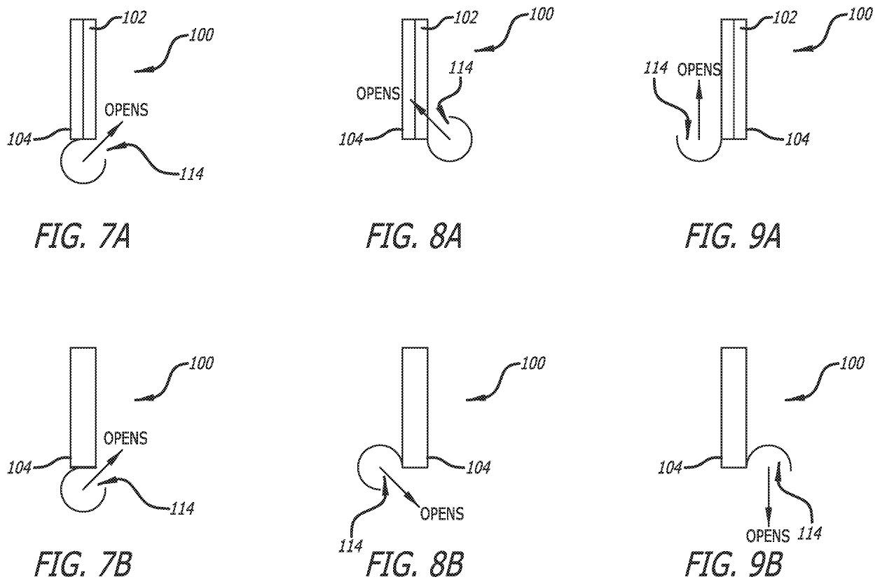

The pair of guidewire clips 110 can be disposed under the back edge 104 of the scalpel blade 100 (see FIGS. 7A and 7B) or along a side of the back edge 104 (see FIGS. 8A and 8B or FIGS. 9A or 9B) of the scalpel blade 100. The pair of guidewire clips 110 are configured to clip onto the guidewire 112 with sufficient clearance for slidably guiding the scalpel blade 100 on top of the guidewire 112 or along the guidewire 112 when skin-nicking a patient's skin and fascia around an insertion site established by a percutaneous puncture. Indeed, the pair of guidewire clips 110 assist a clinician in holding the scalpel blade 100 flush against such a guidewire during the skin-nicking, thereby reducing or even eliminating an occurrence of a skin bridge when expanding the insertion site for a CVC or some other medical device.

Each clip of the pair of guidewire clips 110 includes an opening 114 that opens in an opposite direction from that of the other clip of the pair of guidewire clips 110. In an example, each clip of the pair of guidewire clips 110 can include the opening 114 configured to open toward the back edge 104 of the scalpel blade 100 and away from a side of the scalpel blade 100 opposite the other clip of the pair of guidewire clips 110 as shown between at least FIGS. 7A and 7B. In another example, each clip of the pair of guidewire clips 110 can include the opening 114 configured to open into a side of the scalpel blade 100 or opposite thereto on the same side of the scalpel blade 100 as shown between FIGS. 8A and 8B. In another example, each clip of the pair of guidewire clips 110 can include the opening 114 configured to open along a side of the scalpel blade 100 or opposite thereto on the same side of the scalpel blade 100 as shown between FIGS. 9A and 9B. Each clip of the pair of guidewire clips 110 is mutually exclusive of the other clip of the pair of guidewire clips 110 with respect to the opening 114 thereof in the foregoing configurations. Such an arrangement provides a tortured path of escape for the guidewire 112, thereby ensuring the scalpel blade 100 remains clipped on the guidewire 112 once clipped thereon as shown in FIGS. 1-5. Notably, the guidewire 112 is loaded from both sides of the scalpel blade 100 as set forth in the guidewire-loading step of the method set forth below. For this reason, each clip of the pair of guidewire clips 110 is sufficiently separated along the back edge 104 of the scalpel blade 100 from the other clip of the pair of guidewire clips 110 to allow the guidewire 112 to easily cross over the back edge 104 of the scalpel blade 100 without appreciably bending the guidewire 112 when loading or extracting the guidewire 112 from each clip of the pair of guidewire clips 110. Such a configuration for the pair of guidewire clips 110 is notable with respect to loading or extracting the guidewire therefrom because appreciably bending the guidewire 112 can kink the guidewire 112. In addition, overmanipulating the guidewire 112 can lead to loss of the guidewire 112 from a blood-vessel lumen of a patient, which loss could require another percutaneous puncture in order to secure access to the blood-vessel lumen with the guidewire 112.

The pair of guidewire clips 110 can be ‘C’-shaped clips, ranging from approximately a semicircle in transverse cross section up to approximately a three-quarter sector of a circle in transverse cross section. When the guidewire 112 is loaded in the pair of guidewire clips 110, the guidewire 112 is coaxial with the pair of guidewire clips 110. Indeed, the guidewire 112 is sufficiently coaxial with the pair of guidewire clips 110 for rotating the scalpel blade 100—or the scalpel 116 set forth below when the scalpel blade 100 is coupled to the scalpel handle 118—around the guidewire 112 to a desired rotational angle for the skin-nicking of the patient's skin and fascia around the insertion site.

Each clip of the pair of guidewire clips 110 can be metal or plastic such as a thermoplastic. When the pair of guidewire clips 110 are metal, the pair of guidewire clips 110 can be die-cut with the scalpel blade 100 and machined to a desired shape such that the pair of guidewire clips 110 are integral with a remainder of the scalpel blade 100. Alternatively, the pair of guidewire clips 110 can be die-cut separately from the scalpel blade 100, machined to a desired shape, and coupled to the scalpel blade 100 such as by welding (e.g., microwelding, laser welding, etc.) or riveting through through holes die-cut or drilled into both the scalpel blade 100 and the pair of guidewire clips 110. When the pair of guidewire clips 110 are plastic, the pair of guidewire clips 110 can be molded such that each clip of the pair of guidewire clips 110—in addition to being appropriately shaped—includes a roll-up or meltable rivet shank extending therefrom. Such a roll-up or meltable rivet shank can be inserted into a corresponding through hole die-cut or drilled into the scalpel blade 100 and respectively rolled up by way of, for example, a rivet press or melted by way of, for example, a sonotrode of an ultrasonic welder.

The handle-attachment slot 106 is configured for attaching the scalpel handle 118 to the scalpel blade 100 to form the scalpel 116 set forth below. Such a handle-attachment slot can be die-cut with the scalpel blade 100.

### Scalpels

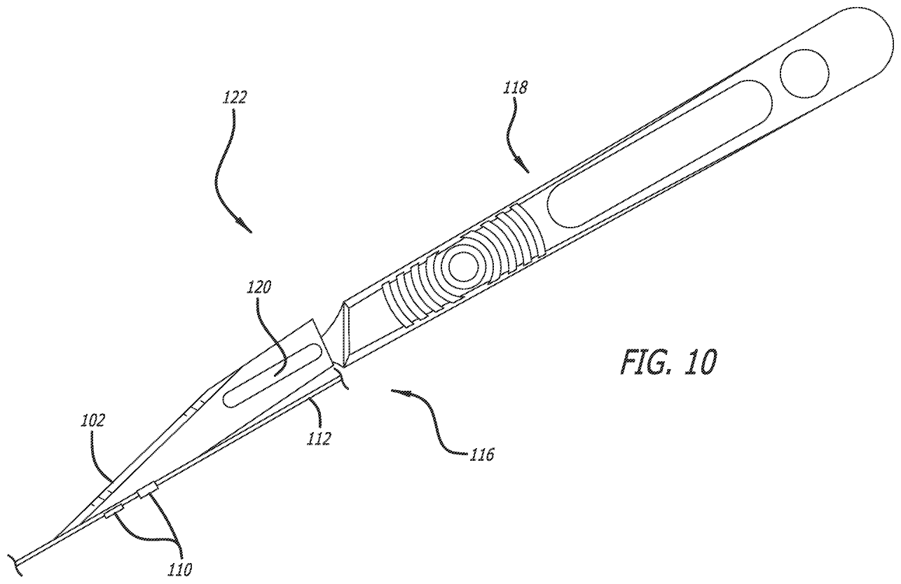

FIG. 10 illustrates a scalpel 116 in accordance with some embodiments.

As shown, the scalpel 116 includes a scalpel handle 118 and the scalpel blade 100 coupled to the scalpel handle 118.

The scalpel handle 118 includes a blade-attachment fixture 120. The blade-attachment fixture 120 is configured to be disposed in the handle-attachment slot 106 such as inserted into the handle-attachment slot 106 and proximally slid in the handle-attachment slot 106 until seated. Indeed, FIG. 10 shows the blade-attachment fixture 120 disposed in the handle-attachment slot 106 coupling the scalpel blade 100 to the scalpel handle 118 to form the scalpel 116. Such a scalpel handle can be formed of surgical steel.

### Scalpel Assemblies

FIG. 10 illustrates a scalpel assembly 122 in accordance with some embodiments.

As shown, the scalpel assembly 122 includes the guidewire 112 and the scalpel 116 with the pair of guidewire clips 110 of the scalpel blade 100 clipped onto the guidewire 112. However, other assemblies are possible such as a scalpel-blade assembly including the guidewire 112 and the scalpel blade 100 with the pair of guidewire clips 110 of the scalpel blade 100 clipped onto the guidewire 112. (See FIGS. 1-5 for such a scalpel-blade assembly.)

The scalpel assembly 122 or even the scalpel-blade assembly can be provided in a kit in a protected, but ready-to-use state, for example, with a plastic scalpel-blade shield over the blade edge 102 of the scalpel blade 100 and a pair of stops such as a pair of doubled-over sticker tabs over the guidewire 112 flanking the pair of guidewire clips 110 and stopping any sliding of the scalpel blade 100 over the guidewire 112. Such a ready-to-use scalpel assembly is useful with at least a slotted needle, which includes a side slot from which the guidewire 112 can be easily pulled out of following a percutaneous puncture with the needle. Alternatively, such as when a more traditional, non-slotted needle is used for the percutaneous puncture, the scalpel assembly 122 or the scalpel-blade assembly is assembled after the needle-withdrawing step of the method set forth below. This is because the more traditional, non-slotted needle needs to be withdrawn over a proximal end of the guidewire 112, which obviates use of the scalpel assembly 122 or scalpel-blade assembly in the foregoing ready-to-use state with at least the scalpel blade 100 already clipped onto the guidewire 112.

### Methods

Methods include a method for inserting a medical device such as a catheter (e.g., CVC) into a blood-vessel lumen, which utilizes a skin-nicking step with the scalpel blade 100. Such a method includes one or more steps selected from a needle tract-establishing step, a guidewire-inserting step, a needle-withdrawing step, a scalpel-forming step, a guidewire-loading step, a scalpel- or scalpel blade-rotating step, the skin-nicking step, a scalpel- or scalpel blade-removing step, and a catheter-inserting step.

The needle tract-establishing step includes establishing a needle tract from an area of skin to the blood-vessel lumen of a patient with a needle at a chosen insertion site for inserting the catheter.

The guidewire-inserting step includes inserting the guidewire 112 through the needle into the blood-vessel lumen.

The needle-withdrawing step includes withdrawing the needle from the needle tract leaving the guidewire 112 in place in the blood-vessel lumen. As set forth above, if the needle is a slotted needle the guidewire 112 can be easily pulled out of the side slot of the slotted needle immediately after withdrawing the needle from the needle tract; however, if the needle is the more traditional, non-slotted needle, the needle needs to be withdrawn over the proximal end of the guidewire 112.

The scalpel-forming step is performed if the scalpel 116 is not already formed like that of the scalpel assembly 122 set forth above, for example. The scalpel-forming step includes coupling the scalpel blade 100 to the scalpel handle 118 to form the scalpel 116 by inserting the blade-attachment fixture 120 of the scalpel handle 118 into the handle-attachment slot 106 of the scalpel blade 100.

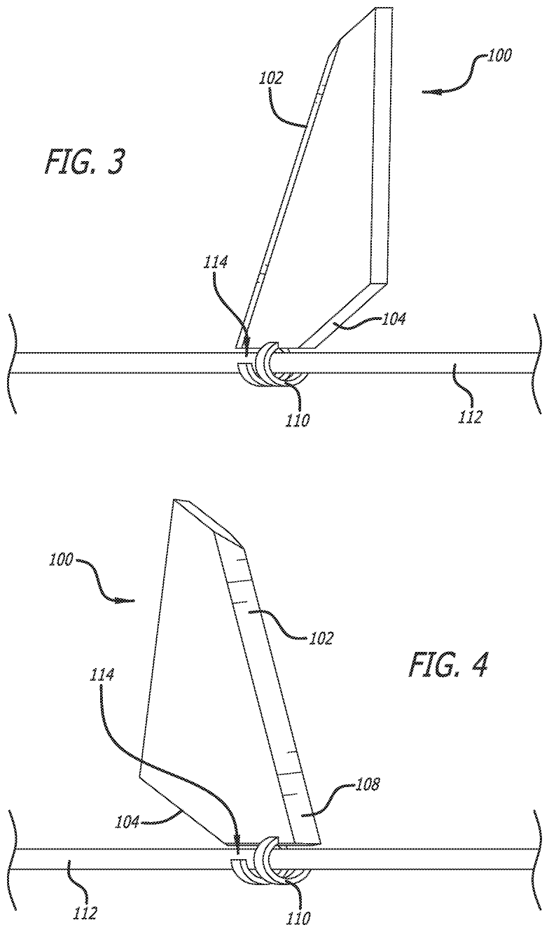

The guidewire-loading step is performed if the scalpel blade 100 is not already clipped onto the guidewire 112 like that of the scalpel assembly 122 set forth above, for example. The guidewire-loading step includes loading the guidewire 112 into the pair of guidewire clips 110 of the scalpel blade 100 by rotating the scalpel blade 100 and placing the guidewire 112 in the opening 114 of each clip of the pair of guidewire clips 110 for each clip of the pair of guidewire clips 110. Indeed, the guidewire-loading step includes rotating the scalpel blade 100 in hand as convenient for access to the opening 114 of a distal clip of the pair of guidewire clips 110, loading the guidewire 112 into the opening 114 of the distal clip, rotating the scalpel blade 100 around the guidewire 112 as convenient for access to the opening 114 of a proximal clip of the pair of guidewire clips 110, and loading the guidewire 112 into the opening 114 of the proximal clip. (See FIGS. 3 and 4 with the understanding that any order of loading the guidewire 112 into the pair of guidewire clips 110 of the scalpel blade 100 is available.) Again, the opening 114 of each clip of the pair of guidewire clips 110 opens on opposite sides of the scalpel blade 100, which calls for rotating the scalpel blade 100 through opposite rotation angles when rotating the scalpel blade 100 and placing the guidewire 112 in the opening 114 of each clip of the pair of guidewire clips 110 for each clip of the pair of guidewire clips 110.

The scalpel- or scalpel blade-rotating step includes rotating the scalpel 116 or the scalpel blade 100 around the guidewire 112 to a desired rotational angle for the skin-nicking of the skin and fascia of the patient around the insertion site. Again, the guidewire 112 is sufficiently coaxial with the pair of guidewire clips 110 for the rotating of the scalpel 116 or the scalpel blade 100 around the guidewire 112.

The skin-nicking step includes skin-nicking skin and fascia of the patient around the insertion site with the scalpel blade 100 guided along the guidewire 112.

The scalpel- or scalpel blade-removing step includes removing the scalpel 116 or the scalpel blade 100 from the guidewire 112. Indeed, the scalpel- or scalpel blade-removing step includes unloading the guidewire 112 from the pair of guidewire clips 110 of the scalpel blade 100 by rotating the scalpel blade 100 and extracting the guidewire 112 from the opening 114 of each clip of the pair of guidewire clips 110 for each clip of the pair of guidewire clips 110. However, the scalpel- or scalpel blade-removing step can alternatively include withdrawing the scalpel 116 or the scalpel blade 100 over the proximal end of the guidewire 112.

The catheter-inserting step includes inserting the catheter into the blood-vessel lumen over the guidewire 112.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. A scalpel blade, comprising:
a blade edge terminating with a blade tip at a distal end of the scalpel blade; and
a back edge including a pair of guidewire clips configured to clip onto a guidewire with sufficient clearance for slidably guiding the scalpel blade along the guidewire when skin-nicking a patient's skin and fascia around an insertion site established by a percutaneous puncture.

2. The scalpel blade according to claim 1, wherein the pair of guidewire clips are disposed under the back edge of the scalpel blade or along a side of the back edge of the scalpel blade.

3. The scalpel blade according to claim 1, wherein each clip of the pair of guidewire clips includes an opening that opens toward the back edge of the scalpel blade and away from a side of the scalpel blade opposite the other clip of the pair of guidewire clips.

4. The scalpel blade according to claim 3, wherein each clip of the pair of guidewire clips is approximately a three-quarter sector of a circle in transverse cross section.

5. The scalpel blade according to claim 1, wherein each clip of the pair of guidewire clips includes an opening that opens along a side of the scalpel blade or opposite thereto on a same side of the scalpel blade, each clip of the pair of guidewire clips mutually exclusive of the other clip of the pair of guidewire clips with respect to the opening thereof.

6. The scalpel blade according to claim 5, wherein each clip of the pair of guidewire clips is approximately a semicircle in transverse cross section.

7. The scalpel blade according to claim 1, wherein each clip of the pair of guidewire clips is sufficiently separated along the back edge of the scalpel blade from the other clip of the pair of guidewire clips to allow the guidewire to easily cross over the back edge of the scalpel blade without appreciably bending the guidewire when loading the guidewire into each clip of the pair of guidewire clips.

8. The scalpel blade according to claim 1, wherein the guidewire is coaxial with the pair of guidewire clips when the guidewire is loaded therein.

9. The scalpel blade according to claim 8, wherein the guidewire is sufficiently coaxial with the pair of guidewire clips for rotating the scalpel blade around the guidewire to a desired rotational angle for the skin-nicking of the patient's skin and fascia around the insertion site.

10. The scalpel blade according to claim 1, further comprising a handle-attachment slot configured for attaching a scalpel handle to the scalpel blade to form a scalpel.

11. A scalpel, comprising:
a scalpel handle; and
a scalpel blade coupled to the scalpel handle, the scalpel blade including:
a blade edge terminating with a blade tip at a distal end of the scalpel blade; and
a back edge including a pair of guidewire clips configured to clip onto a guidewire with sufficient clearance for slidably guiding the scalpel along the guidewire when skin-nicking a patient's skin and fascia around an insertion site established by a percutaneous puncture.

12. The scalpel according to claim 11, the scalpel handle including a blade-attachment fixture and the scalpel blade further including a handle-attachment slot, the blade-attachment fixture disposed in the handle-attachment slot coupling the scalpel blade to the scalpel handle.

13. The scalpel according to claim 11, wherein the pair of guidewire clips are disposed under the back edge of the scalpel blade or along a side of the back edge of the scalpel blade.

14. The scalpel according to claim 11, wherein each clip of the pair of guidewire clips includes an opening that opens toward the back edge of the scalpel blade and away from a side of the scalpel blade opposite the other clip of the pair of guidewire clips.

15. The scalpel according to claim 14, wherein each clip of the pair of guidewire clips is approximately a three-quarter sector of a circle in transverse cross section.

16. The scalpel according to claim 11, wherein each clip of the pair of guidewire clips includes an opening that opens along a side of the scalpel blade or opposite thereto on a same side of the scalpel blade, each clip of the pair of guidewire clips mutually exclusive of the other clip of the pair of guidewire clips with respect to the opening thereof.

17. The scalpel according to claim 16, wherein each clip of the pair of guidewire clips is approximately a semicircle in transverse cross section.

18. The scalpel according to claim 11, wherein each clip of the pair of guidewire clips is sufficiently separated along the back edge of the scalpel blade from the other clip of the pair of guidewire clips to allow the guidewire to easily cross over the back edge of the scalpel blade without appreciably bending the guidewire when loading the guidewire into each clip of the pair of guidewire clips.

19. The scalpel according to claim 11, wherein the guidewire is coaxial with the pair of guidewire clips when the guidewire is loaded therein.

20. The scalpel according to claim 19, wherein the guidewire is sufficiently coaxial with the pair of guidewire clips for rotating the scalpel around the guidewire to a desired rotational angle for the skin-nicking of the patient's skin and fascia around the insertion site.

21. A scalpel assembly, comprising:
a guidewire;
a scalpel handle; and
a scalpel blade coupled to the scalpel handle to form a scalpel, the scalpel blade including:
a blade edge terminating with a blade tip at a distal end of the scalpel blade; and
a back edge including a pair of guidewire clips clipped onto the guidewire with sufficient clearance for slidably guiding the scalpel along the guidewire when skin-nicking a patient's skin and fascia around an insertion site established by a percutaneous puncture.

22. The scalpel assembly according to claim 21, the scalpel handle including a blade-attachment fixture and the scalpel blade further including a handle-attachment slot, the blade-attachment fixture disposed in the handle-attachment slot coupling the scalpel blade to the scalpel handle.

23. The scalpel assembly according to claim 21, wherein the pair of guidewire clips are disposed under the back edge of the scalpel blade or along a side of the back edge of the scalpel blade.

24. The scalpel assembly according to claim 21, wherein each clip of the pair of guidewire clips includes an opening that opens toward the back edge of the scalpel blade and away from a side of the scalpel blade opposite the other clip of the pair of guidewire clips.

25. The scalpel assembly according to claim 24, wherein each clip of the pair of guidewire clips is approximately a three-quarter sector of a circle in transverse cross section.

26. The scalpel assembly according to claim 21, wherein each clip of the pair of guidewire clips includes an opening that opens along a side of the scalpel blade or opposite thereto on a same side of the scalpel blade, each clip of the pair of guidewire clips mutually exclusive of the other clip of the pair of guidewire clips with respect to the opening thereof.

27. The scalpel assembly according to claim 26, wherein each clip of the pair of guidewire clips is approximately a semicircle in transverse cross section.

28. The scalpel assembly according to claim 21, wherein each clip of the pair of guidewire clips is sufficiently separated along the back edge of the scalpel blade from the other clip of the pair of guidewire clips to allow the guidewire to easily cross over the back edge of the scalpel blade without appreciably bending the guidewire when loading the guidewire into each clip of the pair of guidewire clips.

29. The scalpel assembly according to claim 21, wherein the guidewire is coaxial with the pair of guidewire clips when the guidewire is loaded therein.

30. The scalpel assembly according to claim 29, wherein the guidewire is sufficiently coaxial with the pair of guidewire clips for rotating the scalpel around the guidewire to a desired rotational angle for the skin-nicking of the patient's skin and fascia around the insertion site.

31-40. (canceled)
